(19) United States
(12) Patent Application Publication
(10) Pub. No.: US 2015/0306056 A1
(43) Pub. Date: Oct. 29, 2015

(54) USE OF A DHA ESTER FOR PROPHYLACTIC AND/OR CURATIVE TREATMENT OF DREPANOCYTOSIS

(71) Applicant: PIERRE FABRE MEDICAMENT, Boulogne-Billancourt (FR)

(72) Inventors: Jean-Paul CAUBERE, Semalens (FR); Frédérique LANTOINE-ADAM, Toulouse (FR)

(73) Assignee: PIERRE FABRE MEDICAMENT, Boulogne-Billancourt (FR)

(21) Appl. No.: 14/647,364

(22) PCT Filed: Nov. 27, 2013
(86) PCT No.: PCT/EP2013/074863

§ 371 Date: May 26, 2015

(30) Foreign Application Priority Data
Nov. 27, 2012 (FR) 1261291

(51) Int. Cl. A61K 31/232; A61K 45/06; A61K 31/4406

(52) U.S. Cl. CPC A61K 31/232; A61K 31/4406; A61K 45/06

(57) ABSTRACT

The present invention relates to a docosahexaenoic acid ester including an alcohol selected from among the group made up of nicotinol, panthenol, inositol, isosorbide, and isosorbide mononitrate, or one of the pharmaceutically acceptable salts, enantiomers, diastereoisomers, or mixtures thereof, including racemic mixtures, for the use thereof as a drug for the prophylactic and/or curative treatment of drepanocytosis.

The present invention is directed to the use of a DHA ester for prophylactic and/or curative treatment of drepanocytosis.

Drepanocytosis, also called haemoglobin S disease or sickle-cell disease, is a genetic disease of haemoglobin, the protein ensuring the transport of oxygen in the blood. Drepanocytosis is not a very rare disease. It is particularly frequent in populations in the African sub-Saharan region, the West Indies, India, the Middle East and the Mediterranean region in Greece and Italy in particular. It is estimated that more than 100 million persons are affected over the world. It is the leading genetic disease in France and probably in the world.

Drepanocytosis is due to an anomaly of haemoglobin, the main constituent of red blood cells also called erythrocytes. These are flattened discs having a centre that is thinner than the edges. Their so-called biconcave shape is characteristic imparting great flexibility thereto, this being essential so that they can pass through the narrowest blood capillaries. The erythrocyte membrane is formed of a lipid bilayer of which the central part between the outer and inner surfaces is hydrophobic and contains fatty acids. The adhesion, aggregation and deformability of blood cells are highly impacted by the fatty acid content of their membrane.

Haemoglobin is formed of four chains assembled together. Haemoglobin A, in majority in adults, is formed of two so-called alpha chains and two so-called beta chains. In drepanocytosis the beta chains are abnormal. The haemoglobin formed from abnormal beta chains and normal alpha chains is haemoglobin which “agglomerates” in the red blood cells, the term haemoglobin S is used: an abbreviation of the word “sickle”. A red blood cell is normally in the shape of a disc each side of which is slightly hollowed. In drepanocytosis, the agglomeration of haemoglobin S leads to the red blood cells taking on a sickle or crescent shape especially when the amount of oxygen is reduced. Their deformation is “sickle-shaped” and the deformed red blood cells are termed “sickle cells”. In the blood there is a majority of red blood cells of normal appearance as well as sickle shaped red blood cells. In addition to being deformed, sickle red blood cells are more fragile and more rigid than normal red blood cells. They do not circulate well in the vessels preventing them from fully carrying out their role as oxygen transporter, they haemolyse easily in narrow capillaries. The production of the beta chain of haemoglobin is dependent on two genes, the “beta-globin” genes located on chromosome 31. At molecular level the beta chains are abnormal on account of a glutamic acid at position 6 replaced by valine.

Haemoglobin S differs from haemoglobin A that is normal, through its slower electrophoretic mobility, but above all through the insolubility of its deoxygenated form which easily crystallizes. Haemoglobinosis S is currently the most frequent genetic anomaly in France. A distinction is to be made between the heterozygous forms (A/S), usually silent forms, homozygous forms (S/S) and composite heterozygous forms (essentially S/C, S/beta thalassaemia, S/D-Punjab, S/O-Arab) these being the cause of major drepanocytosis syndromes that are always serious clinically and haematologically.

The severity of drepanocytosis is most variable from one person to another and over time for one same person. The disorder is found in infants but usually shows no sign at birth since the red blood cells of infants still contain 50-90% foetal haemoglobin. The symptoms of this disease may occur as early as the age of two or three months, the time of onset of the beta chain. The three main manifestations are anaemia, vaso-occlusive crises and lesser resistance to some infections.

Anaemia designates lack of haemoglobin and translates as excessive fatigue and feelings of weakness. The red blood cells that are constantly renewed are produced in the centre of the bones in the red bone marrow. From there they pass into the general circulation where they normally remain for 120 days in the blood circulation and are later destroyed in the spleen. In drepanocytosis, since the sickle red blood cells are abnormally fragile, they are easily destroyed causing anaemia. The severity of anaemia varies over time, it may suddenly aggravate in the event of excessive spleen functioning, when the term splenic sequestration is used. The abnormal red blood cells are rapidly eliminated by the body, and more specifically by the spleen. The sickle cells are considered to be abnormal by the spleen which captures (or sequesters) and then eliminates these cells, aggravating anaemia.

Other cells are involved in the physiopathology of vaso-occlusive crises: endothelial cells, reticulocytes, polymorphonuclear neutrophils, blood platelets. Yet mononuclear cells and platelets have an abnormal polyunsaturated fatty acid composition in patients suffering from drepanocytosis.

vaso-occlusive crises or pain crises are manifested by sudden sharp pain. The sickle red blood cells block the circulation at the blood vessels preventing optimal distribution of oxygen throughout the body. This process can occur in different parts of the body (bone, abdomen, kidney, brain, retina . . . ). These crises may be most painful. Pain is the most frequent manifestation of the disease: it can be sudden and transient or chronic. Dehydration, cold, stress, altitude . . . are contributing factors. Any part of the body may be concerned, but osteoarticular pain is the most frequent. Over the long term, bone infraction may occur leading to joint problems. Ocular involvement is also frequent, with the possible onset of intraocular haemorrhage. It may limit the visual field almost completely.

Infections are one of the most frequent complications of drepanocytosis. They may occur throughout the entire lifetime of drepanocytosis sufferers, possibly endangering life in particular in infants and young children. Bacterial infection is likely to spread rapidly may affect sites causing serious infection e.g. meningitis or osteomyelitis. Pneumococci and salmonella are the most frequent bacteria. This increased susceptibility to infection is due to the fact that the spleen, which plays a major role in the defence process against bacteria, is practically always damaged in these patients.

The progress of the disease is most variable. In general, anaemia progresses in the form of “haemolytic crises” helped or triggered by infection. Painful vaso-occlusive crises occur at variable intervals, and are more or less intense. The outcome is better the better the quality and access to care.

At the present time there is no cure for drepanocytosis, it is merely possible to relieve pain during a crisis and best prevent serious infection. Pain crises are the first reason for consultation or hospital admission. Analgesics may be insufficient: in general pain is such that recourse is had to morphine or morphine derivatives (opioids).

The analgesics that can be cited to treat drepanocytosis include non-steroidal anti-inflammatories, paracetamol, codeine, tramadol, buprenorphine, nalbuphine, orphine, fentanyl, hydromorphone, oxycodone. In some cases these treatments are not always sufficient to relieve pain. Oxygen therapy is often given to hospitalised patients for daily inhalation of oxygen-enriched air to increase oxygenation of body organs and hence relieve pain. There is no particular treatment for anaemia. If it becomes more acute due to a splenic sequestration crisis a blood transfusion may be necessary. Medication can be offered to patients suffering from severe drepanocytosis i.e. hydroxyurea (or hydroxycarbamide), a product used for leukaemia. This molecule acts on ribonucleotide reductase. It is the key enzyme for conversion of the four ribonucleotides to deoxyribonucleotides essential for DNA synthesis. This molecule in adults is capable of increasing the production of the haemoglobin that is normally present in the foetus and in minute quantity at birth (haemoglobin F). The forced production of this foetal haemoglobin F allows a reduction in the agglomeration of haemoglobin S. However this molecule does not act on lung or bone infections and does provide protection either against secondary bone disorders. In addition, hydroxyurea is not devoid of adverse effects, such as an impact on male fertility. At the current time, there is only one option for sustainable treatment of the disease: bone marrow graft, the healthy marrow will produce healthy red blood cells. However this procedure is reserved for a very small minority of patients. It is an operation which requires extremely heavy treatment and may lead to serious, potentially fatal complications.

It is therefore clearly apparent that the treatments offered to persons suffering from drepanocytosis are far from being sufficient. There is a major medical need for novel medicinal products having the least possible number of adverse effects since they are directed towards physiologically weakened persons.

The polyunsaturated fatty acids of the Omega 3 series, in particular docosahexaenoic acid (DHA) and eicosapentaenoic acid (EPA), advantageously purified and concentrated in ethyl ester form are known for their potential use in the treatment of some cardiovascular diseases and for modulation of corresponding risk factors. In particular, they are known for the treatment of hyperlipidaemia, hypercholesterolemia and arterial hypertension. Clinical trials conducted with formulations containing a high concentration of DMA ethyl ester in patients having suffered myocardial infarction have exhibited the efficiency thereof in reducing mortality and sudden death. These results were partly attributed to a stabilising effect of the cell membranes of ventricular cardiomyocytes which prevent the onset of malignant arrhythmia in the presence of ischaemic myocytes in patients having suffered infarction or in experimental models which reproduce such conditions. Also, low DHA levels have been associated inter alia with attention deficit hyperactivity disorders (ADHD) and depression and it would seem that DHA supplementation is efficient in the fight against this type of disease. Similarly a high DHA level is said to be correlated with a lesser risk of the onset of dementia. DHA would therefore appear to play a major role in a multitude of pathologies.

In drepanocytosis, lipid homeostasis is modified and red blood cell DHA is reduced (Ren et al., Prostaglandins, leukotrienes and essential fatty acids 72: 415-421, 2005), since the pathology induces strong anaemia. More recently, Ren et al., 2008 (Int J Vitam Nutr Res, 78(3): 139-147) have shown that the omega-3 content differs in its distribution within the lipid bilayer of red blood cells in patients suffering from drepanocytosis. The cause is attributed to increased peroxidation in these patients due to low anti-oxidant capacity. A clinical trial performed in 10 patients showed the advantage of treatment with fish oil in drepanocytosis patients by reducing the number of pain crises but without explaining the mechanism thereof (Tomer et al., Thromb. Haemost. 85(6): 966-974, 2001). Very recently, in a pilot clinical trial (16 patients) the authors showed that 6-month supplementation with DHA+EPA (10 mg+15 mg/kg/D) in drepanocytosis patients reduces the number of vaso-occlusive crises and haemolysis (Okpala et al., APMIS, 119(7): 442-448, 2011).

On the other hand, the authors do not seek to show whether the action is chiefly borne by EPA alone or DHA alone, or whether it is the association of both which is pharmacologically active.

A trial conducted in man (Terano at al., Atherosclerosis, 46(3): 321-331, 1983) allowed the demonstration that the administration of EPA for 4 weeks in 8 healthy volunteers leads to a reduction in blood viscosity and an increase in the deformability of red blood cells. The authors even report a positive correlation between the EPA content in the membranes of red blood cells and the deformability thereof.

Similarly, in another trial (Ide et al., Int. J. Mol. Med. 11(6): 729-732, 2003) conducted in patients suffering from anaemia subsequent to chronic hepatitis C, the authors sought to verify whether EPA supplementation (1800 mg) for 2 months could be beneficial. It was shown that the mean haemoglobin level in these patients was significantly increased after a treatment time of one month in all the patients, and that this increase was due to reduced loss of red blood cells.

In the light of these studies, it would therefore seem that an intake of EPA is responsible for the action demonstrated in patients with drepanocytosis.

Yet the inventors put forward a reverse hypothesis and believe that it is DHA which plays a predominant role in this pathology. An intake of DHA could increase the DHA erythrocyte level in persons with drepanocytosis and hence reduce deterioration of these red blood cells, these cells being the veritable hub for drepanocytosis, but also the deterioration of other cells involved in the physiopathology of drepanocytosis such as endothelial cells, platelets, mononuclear cells.

Group B vitamins or provitamins have the advantages related to their function. In particular, nicotinol is the derivative alcohol of nicotinic acid (vitamin B3). It is quickly converted to nicotinic acid in the human body. Nicotinic acid, also called niacin, is a group B water-soluble vitamin which can be synthesized from tryptophan. Vitamin B3 plays a major role in the release of energy from food but also in the reduction of cholesterol. However, therapeutic doses that are efficient for hypocholesterolemic and hypolipidemic purposes are higher than the amounts synthesized by the body and oral hypocholesterolemic and/or hypotriglyceridemic supplementation proves to be necessary. Vitamin B3 deficiency still exists in some Asian and African countries i.e. in regions where there is a high incidence of drepanocytosis. Since vitamin B3 deficiency leads to general fatigue, an intake of vitamin B3 could be truly beneficial in anaemic patients who already tire more rapidly.

Panthenol is the alcohol derived from pantothenic acid, better known under the name vitamin B5. In the body, panthenol converts to pantothenic acid which then becomes a major portion of the compound “coenzyme A”, which is of particular interest in cell metabolism. It takes part in the metabolism of fats, carbohydrates and proteins. Panthenol also takes part in the formation of acetylcholine and adrenal steroids. It is also involved in the detoxification of foreign bodies and in resistance to infections, this being of particular interest in persons with drepanocytosis.

Inositol or vitamin B7 mobilises fats preventing the accumulation thereof. It also has an anxiolytic effect. It tones the nervous system and liver. It also allows the reducing of cholesterol levels in the blood. It is involved in the increase in serotonin activity, control over intracellular calcium concentration, the maintaining of cell membrane potential and cytoskeleton assembly. Inositol deficiency may lead to muscular pain and eye disease. As a result an intake of inositol can only be beneficial for drepanocytosis patients.

Isosorbide, in particular isosorbide mononitrate is a powerful peripheral vasodilator. It further has diuretic properties relieving the work load of the kidneys, these being a priority target during vaso-occlusive crises; an intake of isosorbide may also be beneficial for persons with drepanocytosis.

It is important to note that vitamins B3 and B5 take part in the production of red blood cells. In persons suffering from drepanocytosis one or other of these vitamins are therefore the preferred alcohols of this invention.

Surprisingly, the inventors have discovered that the administration of an ester of DHA with an alcohol allowed a major increase in DHA levels within the red blood cells.

The subject of the invention is therefore an ester of docosahexaenoic acid with an alcohol selected from the group formed by:

nicotinol having the following formula:

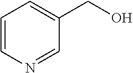

panthenol having the following formula:

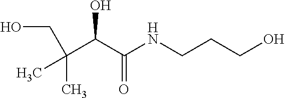

inositol having the following formula:

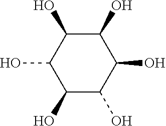 [SMILES: O[C@H]1[C@H](O)[C@@H](O)[C@H](O)[C@@H](O)[C@H]1O]

isosorbide having the following formula:

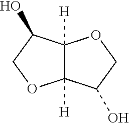

and isosorbide mononitrate having the following formula:

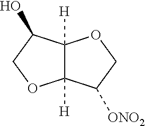

or one of the pharmaceutically acceptable salts thereof, the enantiomers, diastereoisomers, or a mixture thereof including racemic mixtures, for use as medicinal product in the prophylactic and/or curative treatment of drepanocytosis.

Advantageously, the ester of the invention is panthenyl docosahexaenoate or “D-panthenol DHA ester” having the following formula:

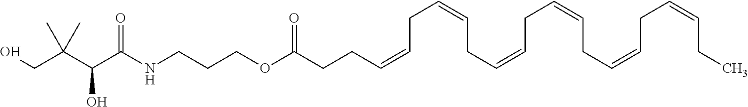 [SMILES: CC/C=C\C/C=C\C/C=C\C/C=C\C/C=C\C/C=C\CCC(=O)OCCCNC(=O)[C@@H](O)C(C)(C)CO]

for use thereof as medicinal product for the prophylactic and/or curative treatment of drepanocytosis.

In one particular embodiment of the invention, the DHA ester with an alcohol selected from the group formed by nicotinol, panthenol, inositol, isosorbide or isosorbide mononitrate is used as medication intended to prevent and/or relieve vaso-occlusive crises in patients suffering from drepanocytosis.

In another particular embodiment, the DHA ester with an alcohol selected from the group formed by nicotinol, panthenol, inositol, isosorbide or isosorbide mononitrate is used as medicinal product intended to prevent and/or treat anaemia in patients suffering from drepanocytosis.

In the present invention, by drepanocytosis is meant all genetic forms of the disease including homozygous and composite heterozygous drepanocytosis.

In the present invention, by prophylactic treatment is meant treatment the objective of which is to prevent the onset or spread of the disease. By curative treatment is meant treatment the objective of which is to cure, minimise or relieve symptoms.

In the present invention by “enantiomers” it is meant to designate optical isomer compounds which have the same molecular formulas but which differ through their spatial configuration and are non-superimposable images in a mirror. By “diastereoisomers” is meant optical isomers which are not images of one another in a mirror. In the meaning of the present invention a “racemic mixture” is a mixture in equal proportions of left-handed and right-handed enantiomers in a chiral molecule.

In the present invention, by “pharmaceutically acceptable” or “acceptable from a pharmaceutical viewpoint” is meant useful for the preparation of a pharmaceutical composition which is generally safe, non-toxic and neither biologically nor otherwise undesirable and is acceptable for veterinary use and for human pharmaceutical use.

By “pharmaceutically acceptable salts” of a compound are meant salts which are pharmaceutically acceptable as defined herein and which have the desired pharmacological activity of the parent compound. Such salts include:

acid addition salts formed with mineral acids such as hydrochloric acid, hydrobromic acid, sulfuric acid, nitric acid, phosphoric acid and the like; or formed with organic acids such as acetic acid, benzenesulfonic acid, benzoic acid, camphorsulfonic acid, citric acid ethane-sulfonic acid, fumaric acid, glucoheptonic acid, gluconic acid, glutamic acid, glycolic acid, hydroxynaphthoic acid, 2-hydroxyethanesulfonic acid, lactic acid, maleic acid, malic acid, mandelic acid, methanesulfonic acid, muconic acid, 2-naphthalenesulfonlic acid, propionic acid, salicylic acid, succinic acid, dibenzoyl-L-tartaric acid, tartaric acid, p-toluenesulfonic acid, trimethylacetic acid, trifluoroacetic acid and the like; or

the salts formed when an acid proton contained in the parent compound is replaced by a metal ion e.g. an alkaline metal ion, an alkaline-earth metal ion or aluminium ion; or is coordinated with an organic or inorganic base. Acceptable organic bases include diethanolamine, ethanolamine, N-methylglucamine, triethanolamine, tromethamine and the like. Acceptable inorganic bases include aluminium hydroxide, calcium hydroxide, potassium hydroxide, sodium carbonate and sodium hydroxide.

The preferred pharmaceutically acceptable salts are the salts formed from hydrochloric acid, trifluoroacetic acid, dibenzoyl-L-tartaric acid and phosphoric acid.

It is to be understood that all the references to pharmaceutically acceptable salts include the solvent addition forms (solvates) or crystalline forms (polymorphs) such as defined herein, of the same acid addition salt.

The present invention further concerns a pharmaceutical composition comprising the DHA ester with an alcohol selected from the group formed by nicotinol, panthenol, inositol, isosorbide or isosorbide mononitrate, and at least one pharmaceutically acceptable excipient, for use thereof as medicinal product in the prophylactic and/or curative treatment of drepanocytosis.

The pharmaceutical composition of the present invention can be used as medicinal product intended to prevent and/or relieve vaso-occlusive crises in patients suffering from drepanocytosis.

The pharmaceutical composition of the present invention can be used as medicinal product intended to prevent and/or treat anaemia in patients suffering from drepanocytosis.

The pharmaceutical composition of the present invention can be administered via oral route or via any other pharmaceutical administration route.

The pharmaceutical composition of the present invention can be formulated for administration to mammals, including man. These compositions are produced so that they can be given via oral, sublingual, subcutaneous, intramuscular, intravenous, transdermal, local or rectal route. In this case, the active ingredient can be administered in unit administration forms, in a mixture with conventional pharmaceutical carriers, to animals or to human beings. Suitable unit administration forms include forms for oral route such as tablets, capsules, powders, granules and oral solutions or suspensions, sublingual and mouth administration forms, subcutaneous, topical, intramuscular, intravenous, intranasal or intraocular administration forms and rectal administration forms.

When a solid composition is prepared in tablet form, the main active ingredient is mixed with a pharmaceutical carrier such as gelatine, starch, lactose, magnesium stearate, talc, gum arabic, silica or the like. The tablets can be coated with sucrose or other suitable materials or they can be treated so that they have sustained or delayed release and continuously release a predetermined amount of active ingredient.

A capsule preparation is obtained by mixing the active ingredient with a diluent (optional step) and pouring the mixture obtained into soft or hard capsules.

A preparation in the form of a syrup or elixir may contain the active ingredient together with a sweetener, antiseptic, and a taste enhancer and suitable colouring agent.

Powders or granules dispersible in water can contain the active ingredient in a mixture with dispersing agents or wetting agents, or suspending agents, and taste enhancers or sweeteners.

For rectal administration recourse is had to suppositories which are prepared with binders which melt at rectal temperature e.g. cocoa butter or polyethylene glycols.

For parenteral (intravenous, intramuscular etc.), intranasal or intraocular administration, use is made of aqueous suspensions, isotonic saline solutions or sterile injectable solutions containing pharmacologically compatible dispersing agents and/or wetting agents.

The active ingredient can also be formulated in the form of microcapsules, optionally with one or more additive carriers.

Advantageously the pharmaceutical composition of the present invention is intended for administration via oral or intravenous route, more advantageously via oral route.

The dosages of the pharmaceutical compositions containing an eater of DHA with an alcohol selected from the group formed by nicotinol, panthenol, inositol, isosorbide or isosorbide mononitrate in the compositions of the invention are adjusted to obtain an amount of active substance that is efficient to obtain the desired therapeutic response for a composition particular to the route of administration. The chosen dosage level is therefore dependent on the desired therapeutic effect, the chosen route of administration, the desired treatment time, the patient's weight, age and gender, the sensitivity of the individual to be treated. Therefore the optimal dosage must be determined as a function of parameters considered to be relevant by the specialist concerned. Preferably the DHA ester is administered in acceptable pharmaceutical compositions in which the daily dose is between 250 mg and 10 g per day, more preferably the daily dose is between 1 and 6 g per day e.g. 1 g, 2 g or 4 g/day. It may be necessary to use a higher dose (called loading dose) at the start of prophylactic and/or curative treatment and subsequently to reduce the dose (maintenance dose) throughout treatment.

The pharmaceutical composition of the present invention may further comprise at least one other active ingredient such as an analgesic and/or hydroxyurea leading to an additional or optionally synergic effect.

The invention will be better understood with reference to the following examples.

### EXAMPLE 1

Effect of nicotinol DHA on the fatty acid composition of plasma and red blood cells in dogs treated via oral route. The objective of this first study was to assay total DHA in the blood (plasma and red blood cells) of dogs given nicotinol DHA via oral route.

Two groups of 10 dogs were used:

Group 1: control group

Group 2: nicotinol DHA at 2 g per day.

All the animals were given either a placebo or nicotinol DHA at 2 g per day via oral route for 28 days. Blood samples were taken at D1 (control), D7, D14, D21 and D28.

The total lipids of the plasma (500 μL) and of the red blood cells (≈500 mg, the weighing of red blood cells is more accurate than measuring volume) were extracted with 4 mL of hexane/isopropanol mixture (2:1, v/v) in an acid medium (3M HCl, 500 μL) in the presence of margaric acid as internal standard (100 μg). After agitation and centrifugation (2000 g, 15 minutes, 10° C.) the organic phase was separated. A second extraction with 2 mL of the same solvent was performed under the same conditions. The organic phases were washed with 2 mL of saline water (9% NaCl). The solvents were evaporated under a stream of nitrogen at 40° C.

The total lipids derived from the plasma and red blood cells were then saponified (1 mL of 0.5M NaOH in methanol, 70° C., 30 minutes) then converted to methyl eaters (1 mL, 14% BF₃ in methanol, 70° C., 15 minutes). After hydrolysio (4 mL 9% NaCl) they were extracted with 4 then 2 mL of pentane. The organic phases were washed with 2 mL of saline water (9% NaCl). The solvents were evaporated under a stream of nitrogen at 40° C. The methyl esters were re-dissolved in 200 μL hexane for the plasma and red blood cells. The extracted fatty acid methyl esters were analysed by gas phase chromatography. The chromatograph (Agilent Technologies 6890N) was equipped with a split injector heated to 260° C. (1:10 division ratio), capillary column (length 60 m, diameter 0.25 mm) with BPX70 stationary phase (70% cyanopropylpolyphenylene-siloxane; thickness 0.25 μm) and flame ionization detector heated to 260° C. (hydrogen: 40 mL/min, air: 450 mL/min). The vector gas was helium (constant flow rate 1.5 mL/min). The temperature of the column was initially 150° C. and brought at a temperature gradient of 1.3° C./min up to 220° C. then 40° C./min to reach 260° C. for 5 minutes. The retention times of standard methyl esters allowed identification of the extracted fatty acid methyl esters.

The DHA was quantitated relative to the internal standard (C17:0) added in known quantity to the sample before extraction of the total lipids. It is expressed in μg/mL for the plasma, in μg/g for the red blood cells. The values are given as a mean f standard deviation (in general n=10). The significant differences are shown by a Student's test with 5% threshold.

The results of the DHA plasma levels in the dogs are summarised in Table 1

TABLE 1
Changes in DHA plasma level during treatment
with nicotinol DHA at 2 g/D.

| | D −1 | | D 7 | | D 14 | | D 21 | | D 28 | |
|---|---|---|---|---|---|---|---|---|---|---|
| DHA | Mn | SD | Mn | SD | Mn | SD | Mn | SD | Mn | SD |
| G 1 | 4.2 | 1.5 | 3.9 | 1.7 | 4.4 | 2.1 | 4.2 | 1.5 | 4.2 | 1.6 |
| G 2 | 3.8 | 1.5 | 22.1 | 7.2 | 23.7 | 10.8 | 27.9 | 7.5 | 32.6 | 11.1 |

The DHA levels are expressed in μg/mL, Mn; mean value; SD: standard deviation; G 1: group 1; G 2: group 2. The differences between the 2 groups are statistically significant irrespective of treatment time.

The DHA plasma levels are equivalent between the 2 groups at the start of the experiment. On the other hand, throughout the course of treatment the DHA plasma content was higher in the “nicotinol DNA” group compared with the control group.

Table 2 gives the DHA levels of the red blood cells.

TABLE 2
Changes in DHA levels of red blood cells during
treatment with nicotinol DHA at 2 g/D.

| | D −1 | | D 7 | | D 14 | | D 21 | | D 28 | |
|---|---|---|---|---|---|---|---|---|---|---|
| DHA | Mn | SD | Mn | SD | Mn | SD | Mn | SD | Mn | SD |
| G 1 | 1.9 | 1.1 | 2.1 | 1.2 | 1.9 | 1.0 | 1.9 | 0.7 | 1.8 | 0.7 |
| G 2 | 1.7 | 1.0 | 4.7 | 2.3 | 7.1 | 1.7 | 8.3 | 3.2 | 8.7 | 2.1 |

The DHA levels are expressed in μg/mL, Mn: mean value; SD: standard deviation; G 1: group 1; G 2: group 2. The differences between the 2 groups are statistically significant irrespective of treatment time.

The DHA levels of the red blood cells are equivalent between the 2 groups at the start of the experiment. On the other hand, throughout the entire length of treatment the DNA content of the red blood cells was higher in the “nicotinol DHA” group compared with the control group.

In dogs therefore, the effect of treatment with nicotinol DHA is significant at every treatment time, nicotinol DHA inducing an increase in plasma DHA but more especially inducing a rise in the DHA level of red blood cells.

### EXAMPLE 2

Incorporation of DHA in the plasma and red blood cells of rats receiving panthenol DNA via oral route. The objective of this study was to assay total DHA in the blood (plasma and red blood cells) of rats given panthenol DHA via oral force-feeding for 7 days.

Three groups of 4 rats (2 male and 2 female) were used:

Group 1: control group (olive oil)

Group 2: panthenol DHA at 300 mg/kg per day.

Group 3: panthenol DHA at 1000 mg/kg per day.

The total lipids of the plasma (500 μL) and red blood cells were extracted with a hexane/isopropanol mixture (3:2, v/v) in an acid medium (3M HCl, 1 mL) in the presence of margaric acid as internal standard. The total lipids from the plasma and red blood cell were saponified (i mL 0.5M NaOH in methanol, 70° C., 30 minutes) then converted to methyl esters (1 mL, 14% BF) in methanol, 70° C., 15 minutes). The fatty acid methyl esters were extracted with pentane then analysed by gas phase chromatography. The chromatograph (Agilent Technologies 6890N) was equipped with a split injector heated to 250° C. (division ratio 1:10) and a capillary column (length 60 m, diameter 0.25 mm) with BPX70 stationary phase (70% cyanopropylpolyphenylene-siloxane; thickness 0.25 μm). The vector gas was helium. The temperature of the column was initially 150° C. then brought at a temperature gradient of 1.3° C./min up to 220° C. and held at 220° C. for 10 minutes. The retention times of standard methyl esters allowed identification of the extracted fatty acid methyl eaters.

The DHA was quantitated in relation to the internal standard (C17:0) added in known quantity to the sample before extraction of the total lipids. It is expressed in μg/mL for plasma, in μg/g for the red blood cells. The values are given as a mean±standard deviation.

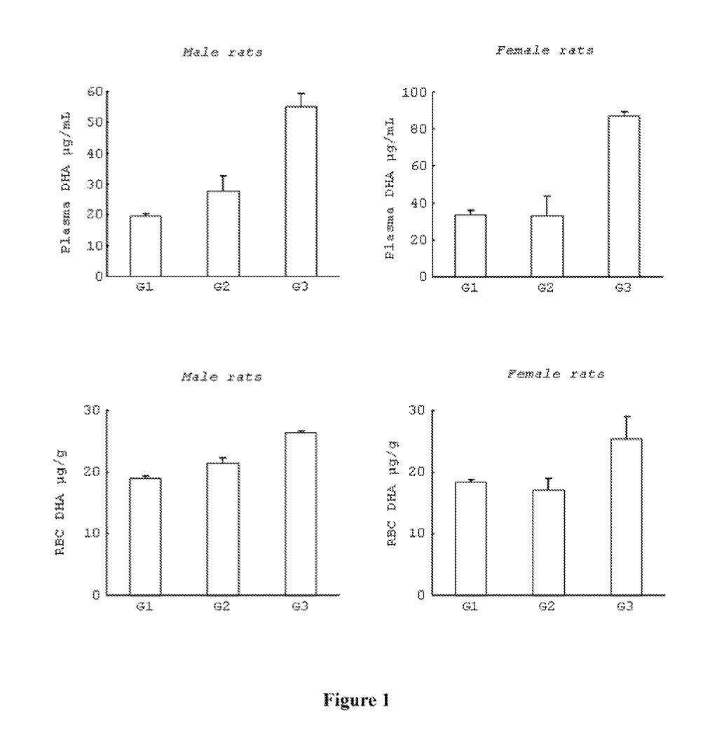

The results of the plasma DHA levels and red blood cell DHA levels in rats are given FIG. 1.

FIG. 1 gives the DHA plasma levels (top diagrams) in male rats (on the left) and in female animals (on the right), and the DHA levels in the red blood cells (bottom diagrams) in the control group (G1), in the rats given panthenol DHA at 300 mg/kg/D (G2) and in the rats given 1000 mg/kg/D panthenol DMA (G3).

For the male rats, the amount of DHA found in the red blood cells and in the plasma was dependent on the dose panthenol DHA given to the animals. In the female rats the amount of DHA found in the red blood cells and in the plasma only increased with the higher dose of panthenol DHA.

DHA panthenol therefore indeed allows release of DHA into the plasma but more especially allows incorporation of DHA in the red blood cells of rate.

### EXAMPLE 3

Concentration of DHA in human red blood cells after absorption of panthenol DHA.

The objective of this clinical trial was to determine total DHA concentrations in the red blood cells of volunteers given a once daily oral dose of panthenol DHA for 28 days. Three doses of panthenol DMA were tested in this trial: 1, 2 and 4 g/day. Twelve persons were included in this trial, 3 were given a placebo (without panthenol DHA) and 9 were given panthenol DHA.

Blood samples were taken before administering panthenol DHA (corresponding to the baseline) and thereafter at days 4, 7, 10 14, 15, 19, 22, 25 and 29 to determine the DHA concentrations in the red blood cells. Two blood samples of 4 mL each were taken in tubes containing EDTA. The tubes were centrifuged at 3000 g for 15 minutes at ambient temperature within 30 minutes after the taking of the sample. The red blood cells were stored at 4° C. and sent to the analysis laboratory under cold storage conditions (2° C. to 8° C.).

The lipids were extracted from the red blood cell samples (≈500 mg) using a hexane/isopropanol mixture (3:2 v/v) in an acid medium in the presence of margaric acid as internal standard (100 μg). The total lipid extracts were saponified and converted to methyl esters. After extraction with pentane, the extracted fatty acid methyl esters were analysed by gas phase chromatography. The chromatograph (Agilent Technologies 6890N) was equipped with a split injector heated to 250° C. and capillary column (length 60 m, diameter 0.25 mm). The vector gas was helium (constant flow rate of 1.5 mL/min). The temperature of the column was initially 150° C. and then brought at a temperature gradient of 1.3° C./min up to 220° C. and held at 220° C. for 10 minutes. The flame ionization detector was heated to 250° C. (hydrogen: 40 mL/min, air: 450 mL/min). The retention times of the standard methyl esters allowed identification of the extracted fatty acid methyl esters.

The DHA was quantitated relative to the internal standard (C17:0) added in known quantity to the sample before extraction of the total lipids. The values are given as a mean±standard deviation.

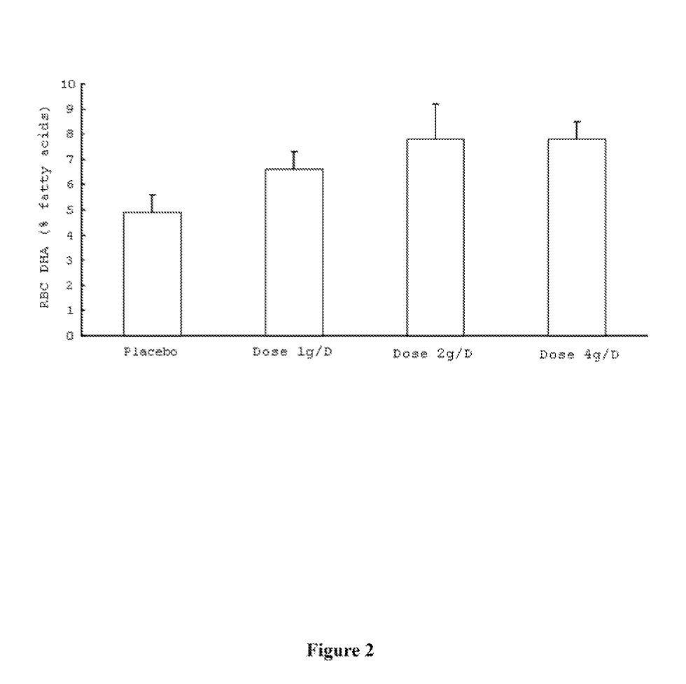

The results of the DHA levels in human red blood cells after administering different doses of panthenol DHA (or placebo) for 28 days are given in FIG. 2. FIG. 2 gives the DHA levels at the end of the trial, calculated as a percentage of fatty acid in human red blood cells as a function of the administered doses of panthenol DHA. Irrespective of the dose of panthenol DHA administered, the DHA level in the red blood cells was increased compared with the placebo group. At a treatment time of 28 days a dose-dependent effect was shown, the maximum effect appearing to be reached with a dose as low as 2 g/day even if variability was lesser with a dose of 4 g/day. The baseline DHA levels (calculated as fatty acid percentage) in human red blood cells in the absence of treatment found in the literature are in the order of 4.8% (Payet et: al. British Journal of Nutrition, 91; 789-796, 20041 Weill et al. Annals of Nutrition & Metabolism, 46: 182-191, 2002) hence very close to the values we found in the placebo group (4.9%). In our treated groups the DHA levels reached 6.6% with 1 g/day of panthenol DNA and 7.8% for the groups given 2 and 4 g/day of panthenol DHA. These differences clearly indicate enriching of the DHA content of human red blood cells with an intake of panthenol DHA.

1. An ester of docosahexaenoic acid with an alcohol selected from the group formed by:
nicotinol having the following formula: 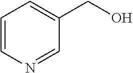
panthenol having the following formula: 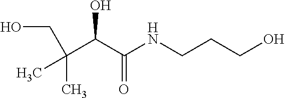
inositol having the following formula: 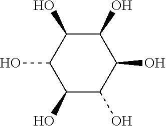 [SMILES: O[C@H]1[C@H](O)[C@@H](O)[C@H](O)[C@@H](O)[C@H]1O]
isosorbide having the following formula: 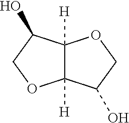
and isosorbide mononitrate having the following formula: 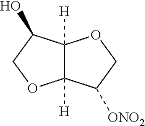
or one of the pharmaceutically acceptable salts thereof, the enantiomers, diastereoisomers or a mixture thereof, including racemic mixtures, for use thereof as medicinal product for the prophylactic and/or curative treatment of drepanocytosis.

2. The eater according to claim 1, having the following formula:

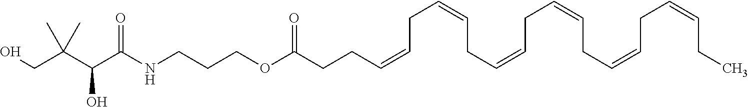 [SMILES: CC/C=C\C/C=C\C/C=C\C/C=C\C/C=C\C/C=C\CCC(=O)OCCCNC(=O)[C@@H](O)C(C)(C)CO]

3. The eater according to claim 1 or 2, for use thereof as medicinal product intended to prevent and/or relieve vaso-occlusive crises in patients suffering from drepanocytosis.

4. The eater according to claim 1 or 2, for use thereof as medicinal product intended to prevent and/or treat anaemia in patients suffering from drepanocytosis.

5. A pharmaceutical composition comprising an eater according to claims 1 to 4 and an excipient acceptable from a pharmaceutical viewpoint for use thereof as medicinal product for the prophylactic and/or curative treatment of drepanocytosis.

6. The pharmaceutical composition according to claim 5, for use thereof as medicinal product intended to prevent and/or relieve vaso-occlusive crises in patients suffering from drepanocytosis.

7. The pharmaceutical composition according to claim 5, for use thereof as medicinal product intended to prevent and/or treat anaemia in patients suffering from drepanocytosis.

8. The pharmaceutical composition according to any of one claims 5 to 7 for administration thereof via oral route.

9. The pharmaceutical composition according to any one of claims 5 to 8, further comprising at least one other active ingredient such as an analgesic and/or hydroxyurea.
